(19) United States
(12) Patent Application Publication
(10) Pub. No.: US 2025/0169736 A1
(43) Pub. Date: May 29, 2025

(54) ADAPTIVE RIGHT LEG DRIVE FOR BIO-POTENTIAL MEASUREMENTS IN THE MRI ENVIRONMENT

(71) Applicant: KONINKLIJKE PHILIPS N.V., EINDHOVEN (NL)

(72) Inventors: BRUCE GEOFFREY APPLETON, ORLANDO, FL (US); PAUL FRANZ REDDER, NEWBERRY, FL (US)

(21) Appl. No.: 18/834,284

(22) PCT Filed: Jan. 16, 2023
(86) PCT No.: PCT/EP2023/050807

§ 371 Date: Jul. 30, 2024

Related U.S. Application Data
(60) Provisional application No. 63/305,805, filed on Feb. 2, 2022.

(51) Int. Cl. A61B 5/305; A61B 5/00; A61B 5/055

(52) U.S. Cl. CPC A61B 5/305; A61B 5/055; A61B 5/7203; A61B 2560/0242

(57) ABSTRACT

The present disclosure describes various systems and methods of adaptively taking bio-potential measurements of within a changing environments. Specifically, the systems and methods are directed to adaptive control of a bio-potential measurement device thereby enabling the use of the bio-potential measurement device within environments having different noise sources, such as environments with and without a device generating strong magnetic fields. Through adaptive measures, the systems and methods of the present disclosure improve the usage of a right leg drive by adaptively changing the nature of the drive itself according to its present environment (e.g., MR vs. non-MR environment). In addition to improvements in common mode rejection (CMR), the systems and methods of the present disclosure can reduce the required analog front-end dynamic range.

### FIELD OF THE DISCLOSURE

The present disclosure is directed generally to systems and methods for taking bio-potential measurements, and more specifically, to systems and methods of adaptively taking bio-potential measurements within a changing environment.

### BACKGROUND

Bio-potential measurements may be acquired as potentials, voltages, and/or electrical fields from a patient's heart (ECG), brain (EEG), and/or muscles (EMG). Because these measurements typically involve voltages at low levels (e.g., microvolts in some cases, millivolts in others, etc.), these bio-potential measurements are susceptible to various noise sources coupling to the body thereby causing interference with the bio-potential signal. Noise sources that may couple onto the body can be from equipment in close proximity to the patient. As a result, these bio-potential signals must be amplified to make them acquirable by medical devices. However, such amplifiers must selectively amplify the desirable signal while rejecting noise and interference signals, such as common mode signals.

Further, within harsh environments, such as magnetic resonance (MR) environments, measurement leads as well as the human body are susceptible to noise pickup during operation of the MR machine (e.g., an MRI). In such environments, conventional implementations for addressing common mode rejection and bio-potential interference fail to function within MR environments for a number reasons and can even make the bio-potential signal integrity worse rather improve it.

### SUMMARY OF THE DISCLOSURE

The present disclosure is directed generally to systems and methods for taking bio-potential measurements, and more specifically, to systems and methods of adaptively taking bio-potential measurements within a changing environment.

According to one example of the present disclosure, provided are systems of adaptively measuring bio-potential signals of a patient in proximity to a noise source. The system can comprise: one or more bio-potential signal sensors for measuring the bio-potential signals of the patient; a driving electrode for applying a driving output signal to the patient; and an adaptive right leg drive (ARLD) circuit operatively connected to the one or more bio-potential signal sensors. The ARLD circuit can comprise a feedback circuit for receiving the bio-potential signals from the patient via the one or more bio-potential signal sensors and outputting a feedback signal, and an ARLD controller having at least one processor and memory storing instructions that, when executed by the at least one processor, performs one or more of the following: receive, from the feedback circuit, the feedback signal; construct the driving output signal; change an operating mode of the ARLD circuit between a first operating mode and a second operating mode; and enable or disable the ARLD circuit.

In an aspect, the one or more bio-potential signal sensors can include at least two electrodes operatively connected to the patient.

In an aspect, the driving electrode can receive the driving output signal from the ARLD circuit and can apply the driving output signal to the patient.

In an aspect, the ARLD controller can further include instructions that, when executed by the at least one processor, perform one or more of the following: receive, via one or more secondary sensors, environmental information; receive environmental configuration information; and receive ARLD control parameters.

In an aspect, the ARLD controller can be operatively connected to a user interface, and at least one of the environmental configuration information and the ARLD control parameters are received via the user interface.

In an aspect, the environmental information can include at least one of local electromagnetic interference (EMI), local audible measurements, local mechanical measurements, and local temperature measurements.

In an aspect, the environmental configuration information can include at least one of mains grid properties, MRI system type, MRI scan to be perform, and lead configurations.

In an aspect, the driving output signal can be constructed by the ARLD controller based on at least one of the environmental information received via the one or more secondary sensors, the environmental configuration information received from the user interface, and the ARLD control parameters received from the user interface.

In an aspect, the ARLD controller can be operatively connected to a user interface, and can further include instructions that, when executed by the at least one processor, perform the following: receive, from the user interface, a user input including one or more user-selectable filters or parameters; and construct the driving output signal based on the user input.

In an aspect, the driving output signal can be constructed by the ARLD controller based on at least the bio-potential signal received from the patient.

In an aspect, the first operating mode of the ARLD circuit can be a non-MR environment mode and the second operating mode of the ARLD circuit can be a MR environment mode.

According to another example of the present disclosure, an adaptive right leg drive (ARLD) circuit is provided. The ARLD circuit can comprise a feedback circuit for receiving bio-potential signals from a patient via one or more bio-potential signal sensors and outputting a feedback signal. The ARLD circuit can further comprise an ARLD controller having at least one processor and memory storing instructions that, when executed by the at least one processor, perform one or more of the following: receive, from the feedback circuit, the feedback signal; construct a driving output signal based on at least the feedback signal; change an operating mode of the ARLD circuit between a first operating mode and a second operating mode; apply, via a driving electrode, the driving output signal; and enable or disable the ARLD circuit.

In an aspect, the ARLD controller can be operatively connected to a user interface, and the ARLD controller can further include instructions that, when executed by the at least one processor, perform one or more of the following: receive, via one or more secondary sensors, environmental information; receive, via the user interface, environmental configuration information; receive, via the user interface, ARLD control parameters; and receive, via the user interface, a user input including one or more user-selectable filters or parameters.

In an aspect, the ARLD controller can construct the driving output signal based on at least one of the feedback signal, environmental information, the environmental configuration information, the ARLD control parameters, and the user input.

In an aspect, the first operating mode of the ARLD circuit can be a non-MR environment mode and the second operating mode of the ARLD circuit can be a MR environment mode.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a block diagram schematic of an adaptive right leg drive system for adaptively measuring biopotential signals from a patient illustrated in accordance with certain aspects of the present disclosure.

FIG. 2 is a block diagram schematic of an adaptive right leg drive system for adaptively measuring biopotential signals from a patient illustrated in accordance with further aspects of the present disclosure.

FIG. 3 is a circuit diagram illustrating a feedback circuit illustrated in accordance with some aspects of the present disclosure.

FIG. 4 is a block diagram of an adaptive right leg drive controller illustrated in accordance with some aspects of the present disclosure.

### DETAILED DESCRIPTION OF EMBODIMENTS

The present disclosure describes various systems and methods of adaptively taking bio-potential measurements of within a changing environment. Specifically, the systems and methods are directed to adaptive control of a bio-potential measurement device thereby enabling the use of the bio-potential measurement device within environments having different noise sources. In particular, existing implementations of a right leg drive fail when placed in an environment with strong magnetic fields (e.g., in a MR environment). Through adaptive measures, the systems and methods of the present disclosure improve the usage of a right leg drive by adaptively changing the nature of the drive itself according to its present environment (e.g., MR vs. non-MR environment). In addition to improvements in common mode rejection (CMR), the systems and methods of the present disclosure can reduce the required analog front-end dynamic range. These and other aspects will be appreciated by those skilled in the art.

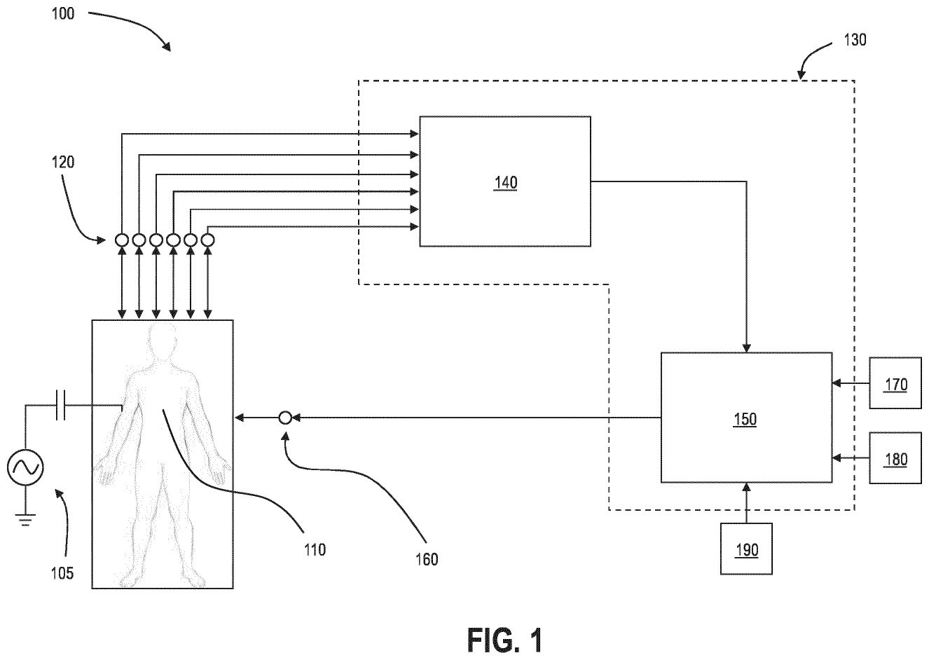

With reference to FIGS. 1-3, these and other aspects of the systems 100 for adaptively measuring biopotential signals from a patient 110 within proximity to a noise source(s) 105 are described. Turning to FIG. 1, a system 100 for adaptively measuring biopotential signals from a patient 110 using an adaptive right leg drive (ARLD) circuit 130 is shown. The system 100 can include one or more biopotential signal sensors 120 configured to take measurements of a biopotential signal(s) from the patient 110, an ARLD circuit 130, and a driving electrode 160 configured to apply a driving output signal to the patient 110.

In an aspect, the ARLD circuit 130 can be configured to receive the biopotential signal measurement(s) from the one or more sensors 120 and construct a driving output signal to be applied to the patient 110 via the driving electrode 160. In particular aspects, the ARLD circuit 130 includes at least a feedback circuit 140 and a ARLD controller 150, the feedback circuit 140 and the ARLD controller being operatively connected to one another. The feedback circuit 140 can be configured to receive the biopotential signal measurement(s) from the one or more sensors 120 and generates a feedback signal that is delivered to the ARLD controller 150, which receives the feedback signal as an input. The ARLD controller 150 can be configured to then construct a driving output signal that is delivered to the patient 110 via at least the driving electrode 160. By actively feeding a driving output signal back onto the body 110, the system 100 is able to cancel out various noise sources coupling to the body 110. Noise sources that couple onto the body are usually from equipment in close proximity and are typically the line frequency (50 Hz/60 Hz). Additionally, the system 100 thereby improves the overall Common Mode Rejection Ratio (CMRR) of the measurement of the biopotential signals.

In further aspects, the system 100 can include one or more additional inputs for adaptively controlling the operation of the system 100, including environmental information 170, environmental configuration information 180, and/or one or more ARLD control parameters 190. In an aspect of the present disclosure, the ARLD controller 150 can receive environmental information 170 that includes one or more of local electromagnetic interference (EMI), local audible measurements, local mechanical measurements, and local temperature measurements. As used herein, the term “local” refers to being in proximity to the patient 110. In certain aspects, the ARLD controller 150 can receive the environmental information 170 from one or more secondary sensors (not shown) operatively connected to the ARLD controller 150.

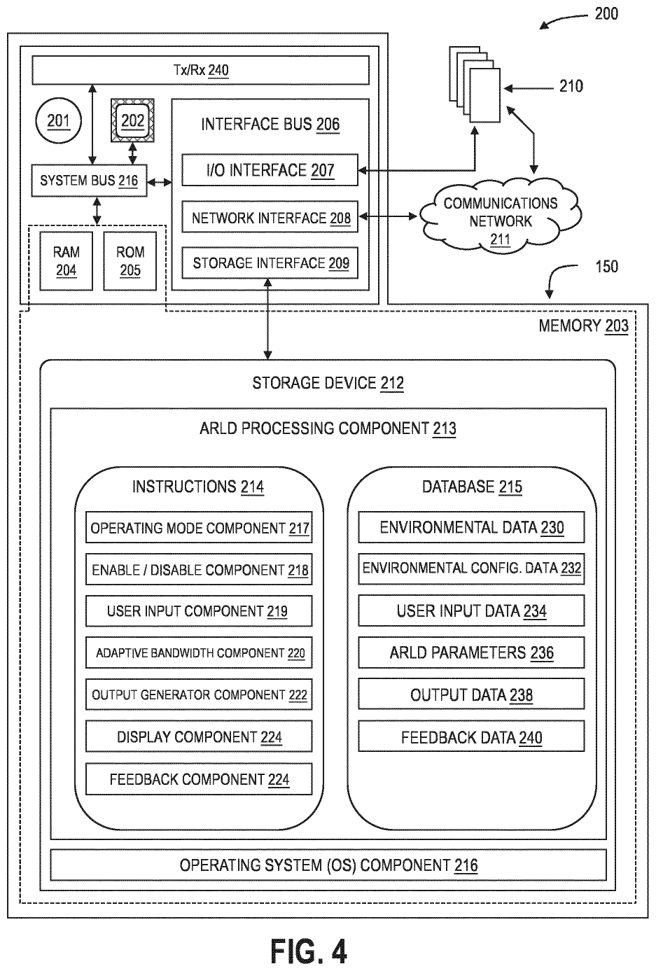

The ARLD controller 150 can also receive environmental configuration information 180 that includes one or more of mains grid properties (Voltage, Frequency), MRI system type (SAR capabilities, Gradient capabilities), MRI scan to be perform (Gradient Frequencies, patterns), and lead configurations (configuration of the one or more sensors 120 and driving electrode 160). In certain aspects, the ARLD controller 150 can receive the environmental configuration information 180 from a peripheral device 210 (see FIG. 4) operatively connected to the ARLD controller 150.

In still further aspects, the ARLD controller 150 can receive ARLD control parameters 190 that includes one or more of operating modes/settings, user-selectable filters or properties, and/or adaptive bandwidth options. In some aspects, the ARLD controller 150 can receive the ARLD control parameters as user input from a peripheral device 210 (see FIG. 4) operatively connected to the ARLD controller 150.

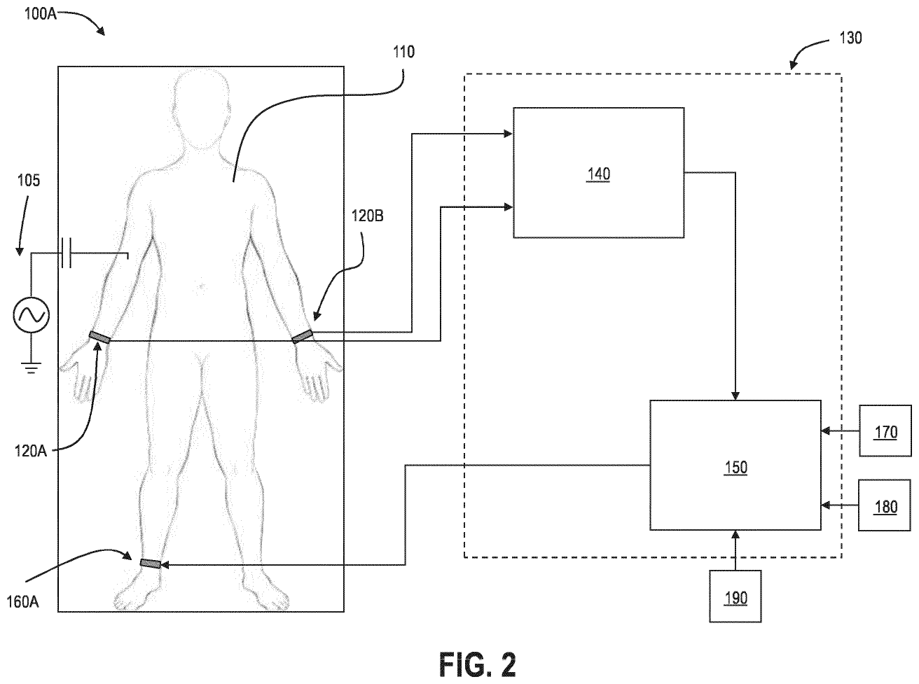

Turning to FIG. 2, a system 100A for adaptively measuring biopotential signals from a patient 110 using an adaptive right leg drive (ARLD) circuit 130 within proximity to a noise source 105 is shown. According to the embodiment shown in FIG. 2, the biopotential signal being measured by the system 100A can be ECG signals. The system 100A can comprise: one or more biopotential signal sensors 120A, 120B, an ARLD circuit 130, and a driving electrode 160A. In specific aspects, the one or more biopotential signal sensors 120A, 120B can include at least a first sensor 120A operatively connected to a first portion of the patient 110 and at least a second sensor 120B operatively connected to a second, different portion of the patient 110. As shown, the first portion of the patient 110 can be a right wrist and the second portion of the patient 110 can be a left wrist. Further, the driving electrode 160 can be operatively connected to a third portion of the patient 110, such as the right leg/ankle of the patient 110. As discussed above, the ARLD circuit 130 can include a feedback circuit 140 and ARLD controller 150 that is configured to construct a driving output signal based at least on the feedback signal, the environmental information 170, the environmental configuration information 180, and the ARLD control parameters 190.

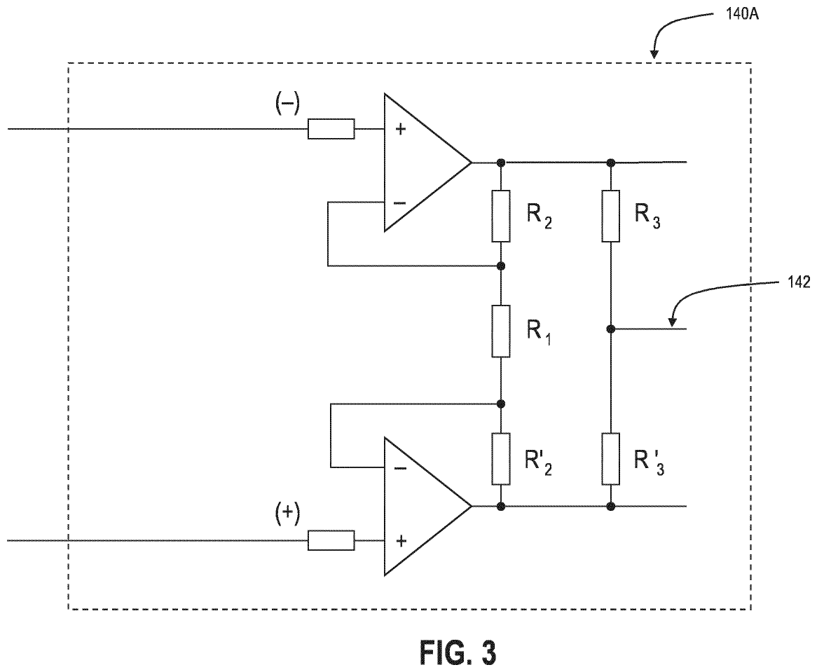

Turning to FIG. 3, a feedback circuit 140A of the ARLD controller 150 is illustrated according to some aspects of the present disclosure. As shown, the feedback circuit 140A includes two operational amplifiers operatively connected with several resistors R₁, R′₁, R₂, R′₂, R₃, and generates a feedback signal 142 that is delivered to the ARLD controller 150.

With reference to FIG. 4, block diagram of an adaptive right leg drive (ARLD) controller 200 is illustrated in accordance with certain aspects of the present disclosure. In certain aspects, the ARLD controller 200 can serve to perform one or more of the following steps: receive, from the feedback circuit 140, the feedback signal; construct a driving output signal based on at least the feedback signal; change an operating mode of the ARLD circuit 130 between a first operating mode and a second operating mode; apply, via a driving electrode 160, the driving output signal; and enable or disable the ARLD circuit 130.

As shown, the ARLD controller 200 can be connected to and/or communicate with one or more peripheral devices 210 and/or a communications network 211 (e.g., local area networks, wide area networks, wireless local area networks, etc.). In particular aspects, the peripheral devices 210 include one or more user interfaces, user input devices, and/or displays. For example, the peripheral devices 210 can include: graphics tablets; joysticks; keyboards; microphones; computer mouse (mice); touch screens (e.g., capacitive, resistive, etc.); trackballs; trackpads; styluses; audio devices; cameras; printers; video devices; and/or the like. According to the present disclosure, the peripheral devices 210 include one or more sensors that are used to measure a biopotential signal associated with a subject 110, as described above.

In certain aspects, the ARLD controller 200 can comprise one or more processors 202 operatively connected to a memory 203 that store instructions 214 for performing one or more of the steps described herein. The memory 203 can include one or more forms of transitory and/or non-transitory memory, including random access memory 204, read only memory 805, and storage device 212. The ARLD controller 200 can include an interface bus 206 can include one or more components that facilitates communication with the peripheral devices 210 and/or communication networks 211, including, but not limited to, an input/output (I/O) interface 207, a network interface 208, and and/or a storage interface 209. The components of the ARLD controller 200 can be interconnected and communicate via a system bus 216. The ARLD controller 200 can include an internal power source 201 and/or be connected to an external power source. The AVT controller 200 can further include transceivers that facilitate wireless communication, including wireless communication between the controller 200 and one or more of the peripheral devices 210.

The memory 203 of the ARLD controller 200 can contain a collection of program and/or database components and/or data such as, but not limited to: operating system component(s) 236 (operating system); an operating mode component 217; an enable/disable component 218; a user input component 219; an adaptive bandwidth component 220; an output generator component 222; a display component 224; and a feedback component 224. These components may be stored and accessed from the storage device(s) 212 accessible through the interface bus 206. In certain aspects, one or more of the components are stored in a local storage device 212. Alternatively, one or more of these components can also be loaded and/or stored in memory 203 via certain peripheral devices, external memory devices, remote storage devices, and the like.

In an aspect, the operating mode component 217 can be a stored program component that, when executed by the at least one processor 202, changes the operating mode of the ARLD circuit 130 from a first operating mode to a second operating mode. For example, the first operating mode of the ARLD circuit 130 can be a non-MR environment mode and the second operating mode of the ARLD circuit 130 can be a MR environment mode, wherein the operating mode component 217 switches between either mode depending on certain pre-defined conditions or inputs (e.g., environmental data 230; environmental configuration data 232; user input data 234; ARLD control parameters 236; feedback data 240; and the like).

In an aspect, the enable/disable component 218 can be a stored program component that, when executed by the at least one processor 202, enables or disables operation of the ARLD circuit 130 based on certain inputs (e.g., environmental data 230; environmental configuration data 232; user input data 234; ARLD control parameters 236; feedback data 240; and the like).

In an aspect, the user input component 219 can be a stored program component that, when executed by at least one processor 202, receives user input 234 from one or more peripheral devices 210 (e.g., user interfaces) and stores the input as user input data 234.

In an aspect, the adaptive bandwidth component 220 can be a stored program component that, when executed by at least one processor 202, will adjust the operating band of interest to maximize the signal of interest or reduce the undesired signal, or a combination of both, based on direct measure information input into controller 150 and/or based on feedback information acquired from one or more of database sources.

In an aspect, the output generator component 222 can be a stored program component that, when executed by at least one processor 202, constructs a driving output signal to be applied to the patient 110 via the driving electrode 160. In particular aspects, the driving output signal can be constructed based on one or more of: environmental data 230; environmental configuration data 232; user input data 234; ARLD control parameters 236; feedback data 240; and the like. Information regarding the constructed output signal can be stored in the memory 203 as output data 238.

In an aspect, the display component 224 can be a stored program component that, when executed by at least one processor 202, displays information related to the ARLD controller 150 on an associated display, including, but not limited to, environmental data 230; environmental configuration data 232; user input data 234; ARLD control parameters 236; output data 238; feedback data 240; and the like.

In an aspect, the feedback component 224 can be a stored program component that, when executed by at least one processor 202, receives and processes the feedback signal 142 from the feedback circuit 140, 140A. In further aspects, the feedback component 224 can, when executed by at least one processor 202, analyze the feedback signal 142 for MR-related features or other interference and/or noise sources, which may be stored in the memory 203 as environmental data 230.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A system of adaptively measuring bio-potential signals of a patient in proximity to a noise source, the system comprising:
one or more bio-potential signal sensors for measuring the bio-potential signals of the patient;
a driving electrode for applying a driving output signal to the patient;
an adaptive right leg drive (ARLD) circuit operatively connected to the one or more bio-potential signal sensors, the ARLD circuit comprising:
a feedback circuit for receiving the bio-potential signals from the patient via the one or more bio-potential signal sensors and outputting a feedback signal; and
an ARLD controller having at least one processor and memory storing instructions that, when executed by the at least one processor, performs one or more of the following:
receive, from the feedback circuit, the feedback signal;
construct the driving output signal;
change an operating mode of the ARLD circuit between a first operating mode and a second operating mode; and
enable or disable the ARLD circuit.

2. The system of claim 1, wherein the one or more bio-potential signal sensors include at least two electrodes operatively connected to the patient.

3. The system of claim 1, wherein the driving electrode receives the driving output signal from the ARLD circuit and applies the driving output signal to the patient.

4. The system of claim 1, wherein the ARLD controller further includes instructions that, when executed by the at least one processor, performs one or more of the following:
receive, via one or more secondary sensors, environmental information;
receive environmental configuration information; and
receive ARLD control parameters.

5. The system of claim 4, wherein the ARLD controller is operatively connected to a user interface, and at least one of the environmental configuration information or the ARLD control parameters is received via the user interface.

6. The system of claim 1, wherein the environmental information includes at least one of local electromagnetic interference (EMI), local audible measurements, local mechanical measurements, or local temperature measurements.

7. The system of claim 4, wherein the environmental configuration information includes at least one of mains grid properties, MRI system type, MRI scan to be perform, or lead configurations.

8. The system of claim 5, wherein the output signal is constructed by the ARLD controller based on at least one of the environmental information received via the one or more secondary sensors, the environmental configuration information received from the user interface, or the ARLD control parameters received from the user interface.

9. The system of claim 1, wherein the ARLD controller is operatively connected to a user interface, and further includes instructions that, when executed by the at least one processor, performs the following:
receive, from the user interface, a user input including one or more user-selectable filters or parameters; and
construct the driving output signal based on the user input.

10. The system of claim 1, wherein the output signal is constructed by the ARLD controller based on at least the bio-potential signal received from the patient.

11. The system of claim 1, wherein the first operating mode of the ARLD circuit is a non-MR environment mode and the second operating mode of the ARLD circuit is a MR environment mode.

12. An adaptive right leg drive (ARLD) circuit, the ARLD circuit comprising:
a feedback circuit for receiving bio-potential signals from a patient via one or more bio-potential signal sensors and outputting a feedback signal; and
an ARLD controller having at least one processor and memory storing instructions that, when executed by the at least one processor, performs one or more of the following:
receive, from the feedback circuit, the feedback signal;
construct a driving output signal based on at least the feedback signal;
change an operating mode of the ARLD circuit between a first operating mode and a second operating mode;
apply, via a driving electrode, the driving output signal; and
enable or disable the ARLD circuit.

13. The ARLD circuit of claim 12, wherein the ARLD controller is operatively connected to a user interface, and the ARLD controller further includes instructions that, when executed by the at least one processor, performs one or more of the following:
receive, via one or more secondary sensors, environmental information;
receive, via the user interface, environmental configuration information;
receive, via the user interface, ARLD control parameters; and
receive, via the user interface, a user input including one or more user-selectable filters or parameters.

14. The ARLD circuit of claim 13, wherein ARLD controller constructs the driving output signal based on at least one of the environmental information, the environmental configuration information, the ARLD control parameters, or the user input.

15. The ARLD circuit of claim 14, wherein the first operating mode of the ARLD circuit is a non-MR environment mode and the second operating mode of the ARLD circuit is a MR environment mode.
